(19) United States
(12) Patent Application Publication
(10) Pub. No.: US 2025/0144982 A1
(43) Pub. Date: May 8, 2025

(54) ACTIVE INGREDIENT GENERATION DEVICE

(71) Applicant: Panasonic Automotive Systems Co., Ltd., Kanagawa (JP)

(72) Inventor: Masahiro JIGI, Fukui (JP)

(21) Appl. No.: 19/015,156

(22) Filed: Jan. 9, 2025

Related U.S. Application Data
(63) Continuation of application No. PCT/JP2023/041593, filed on Nov. 20, 2023.

(30) Foreign Application Priority Data
Dec. 5, 2022 (JP) 2022-194350

(51) Int. Cl. B60H 3/00; F21W 106/00

(52) U.S. Cl. CPC B60H 3/0014; B60H 2003/0042; F21W 2106/00

(57) ABSTRACT

An active ingredient generation device includes: a body; a first air blower that generates a first air stream; an active ingredient generator that generates an active ingredient; a first flow channel through which the first air stream generated by the first air blower flows; and a second flow channel through which the active ingredient generated by the active ingredient generator flows. The body includes: a first blowoff port from which the first air stream flowing through the first flow channel is blown out; and a second blowoff port from which the active ingredient flowing through the second flow channel is blown out. The second blowoff port is disposed at a location where the active ingredient blown out from the second blowoff port is drawn by the first air stream blown out from the first blowoff port.

### CROSS REFERENCE TO RELATED APPLICATION

This is a continuation application of PCT International Patent Application No. PCT/JP2023/041593 filed on Nov. 20, 2023, designating the United States of America, which is based on and claims priority of Japanese Patent Application No. 2022-194350 filed on Dec. 5, 2022.

### FIELD

The present disclosure relates to an active ingredient generation device.

### BACKGROUND

PTL 1 discloses an air cleaner for a vehicle that includes air cleaning means.

### CITATION LIST

### Patent Literature

PTL 1: Japanese Unexamined Patent Application Publication No. 2007-45185

### SUMMARY

However, the air cleaner for a vehicle according to PTL 1 described above can be improved upon.

In view of this, the present disclosure provides an active ingredient generation device capable of improving upon the above related art.

An active ingredient generation device according to an aspect of the present disclosure is an active ingredient generation device that is attached to a ceiling of an interior of a vehicle. The active ingredient generation device includes: a body; a first air blower that is disposed inside the body and generates a first air stream; an active ingredient generator that is disposed inside the body and generates an active ingredient; a first flow channel through which the first air stream generated by the first air blower flows, the first flow channel being disposed inside the body; and a second flow channel through which the active ingredient generated by the active ingredient generator flows, the second flow channel being disposed inside the body. The body includes: a first blowoff port from which the first air stream flowing through the first flow channel is blown out, the first blowoff port being in communication with the first flow channel and an outside of the body; and a second blowoff port from which the active ingredient flowing through the second flow channel is blown out, the second blowoff port being in communication with the second flow channel and the outside of the body. The second blowoff port is disposed at a location where the active ingredient blown out from the second blowoff port is drawn by the first air stream blown out from the first blowoff port.

The active ingredient generation device according to the present disclosure can improve upon the above related art.

### BRIEF DESCRIPTION OF DRAWINGS

These and other advantages and features of the present disclosure will become apparent from the following description thereof taken in conjunction with the accompanying drawings that illustrate a specific embodiment of the present disclosure.

FIG. 1 is a side view of an active ingredient generation device according to Embodiment 1 and its surroundings.

FIG. 2 is an exploded perspective view of the active ingredient generation device in FIG. 1 viewed from diagonally above.

FIG. 3 is an exploded perspective view of the active ingredient generation device in FIG. 1 viewed from diagonally below.

FIG. 4 is a partial cross-sectional view of the active ingredient generation device in FIG. 1.

FIG. 5 is a cross-sectional view of the active ingredient generation device taken along line V-V in FIG. 1.

FIG. 6 is a perspective cross-sectional view of the active ingredient generation device taken along line V-V in FIG. 1.

FIG. 7 is a cross-sectional view illustrating an active ingredient generation device according to Embodiment 2.

FIG. 8 is a perspective view illustrating a part of an active ingredient generation device according to Embodiment 3.

FIG. 9 is a perspective view illustrating a part of an active ingredient generation device according to Embodiment 4.

### DESCRIPTION OF EMBODIMENTS

An active ingredient generation device according to an aspect of the present disclosure is an active ingredient generation device that is attached to a ceiling of an interior of a vehicle. The active ingredient generation device includes: a body; a first air blower that is disposed inside the body and generates a first air stream; an active ingredient generator that is disposed inside the body and generates an active ingredient; a first flow channel through which the first air stream generated by the first air blower flows, the first flow channel being disposed inside the body; and a second flow channel through which the active ingredient generated by the active ingredient generator flows, the second flow channel being disposed inside the body. The body includes: a first blowoff port from which the first air stream flowing through the first flow channel is blown out, the first blowoff port being in communication with the first flow channel and an outside of the body; and a second blowoff port from which the active ingredient flowing through the second flow channel is blown out, the second blowoff port being in communication with the second flow channel and the outside of the body. The second blowoff port is disposed at a location where the active ingredient blown out from the second blowoff port is drawn by the first air stream blown out from the first blowoff port.

Accordingly, the second blowoff port is provided at a location where the active ingredient blown out from the second blowoff port is drawn by the first air stream blown out from the first blowoff port. Therefore, the active ingredient blown out from the second blowoff port can be drawn by the first air stream blown out from the first blowoff port without merging, in the body, the first flow channel with the second flow channel. Therefore, the active ingredient can be efficiently supplied to the interior of the vehicle by the action of the first air stream while avoiding the complication or the like of the structure arising from merging of the first flow channel and the second flow channel in the body.

That is, while the in-vehicle air cleaner according to PTL 1 described above is required to efficiently supply the active ingredient to the interior of the vehicle, the active ingredient generation device according to this aspect of the present disclosure can efficiently supply the active ingredient to the interior of the vehicle.

Moreover, in the active ingredient generation device according to an aspect of the present disclosure, the second blowoff port may be disposed at a location where the active ingredient flowing through the second flow channel is blown out from the second blowoff port by being drawn by the first air stream blown out from the first blowoff port.

Accordingly, the second blowoff port is provided at a location where the active ingredient flowing through the second flow channel is blown out from the second blowoff port by being drawn by the first air stream blown out from the first blowoff port. Therefore, the active ingredient flowing through the second flow channel can be blown out from the second blowoff port by the action of the first air stream without merging, in the body, the first flow channel with the second flow channel. Therefore, the active ingredient can be more efficiently supplied to the interior of the vehicle while avoiding the complication or the like of the structure arising from merging of the first flow channel and the second flow channel in the body.

Moreover, in the active ingredient generation device according to an aspect of the present disclosure, a flow velocity of the first air stream blown out from the first blowoff port is higher than a flow velocity of the active ingredient blown out from the second blowoff port.

Accordingly, the active ingredient is more easily drawn by the first air stream, and therefore, the active ingredient can be more efficiently supplied to the interior of the vehicle.

Moreover, in the active ingredient generation device according to an aspect of the present disclosure, a width of the first blowoff port may be smaller than a width of the first flow channel.

Accordingly, the flow velocity of the first air stream blown out from the first blowoff port can be increased, and the active ingredient is more easily drawn by the first air stream, and therefore, the active ingredient can be more efficiently supplied to the interior of the vehicle.

Moreover, in the active ingredient generation device according to an aspect of the present disclosure, the body includes a plurality of first blowoff ports each being the first blowoff port, a pair of wall portions that guide the first air stream to one of the plurality of first blowoff ports are arranged inside the body, and a distance between the pair of wall portions may decrease toward the one of the plurality of first blowoff ports.

Accordingly, the first air stream can be more efficiently blown out from the first blowoff port, and the active ingredient can be more efficiently drawn by the first air stream, and therefore, the active ingredient can be more efficiently supplied to the interior of the vehicle.

Moreover, in the active ingredient generation device according to an aspect of the present disclosure, the one of the plurality of first blowoff ports may be closest to the second blowoff port, among the plurality of first blowoff ports.

Accordingly, the first air stream can be more efficiently blown out from the first blowoff port that is the closest to the second blowoff port among the plurality of first blowoff ports, and the active ingredient can be more efficiently drawn by the first air stream, and therefore, the active ingredient can be more efficiently supplied to the interior of the vehicle.

Moreover, in the active ingredient generation device according to an aspect of the present disclosure, the body includes a plurality of first blowoff ports each being the first blowoff port, and the plurality of first blowoff ports may have different widths.

Accordingly, the first air streams blown out from the plurality of first blowoff ports can have different flow velocities so that the first air streams can be blown out in a desired direction as a whole, and therefore, the active ingredient can be blown out in a desired direction and can be more efficiently supplied to the interior of the vehicle.

Moreover, in the active ingredient generation device according to an aspect of the present disclosure, the active ingredient generation device includes a plurality of first flow channels each being the first flow channel and a plurality of second flow channels each being the second flow channel. The body includes: the plurality of first blowoff ports from which first air streams flowing through the plurality of first flow channels are blown out, the plurality of first blowoff ports being in communication with the plurality of first flow channels and the outside of the body, the first air streams each being the first air stream; and a plurality of second blowoff ports from which the active ingredient flowing through the plurality of second flow channels is blown out, the plurality of second blowoff ports being in communication with the plurality of second flow channels and the outside of the body, the plurality of second blowoff ports each being the second blowoff port. The plurality of second blowoff ports may be arranged at locations where the active ingredient flowing through the plurality of second flow channels that are in communication with the plurality of second blowoff ports is blown out from the plurality of second blowoff ports by being drawn by the first air streams blown out from the plurality of first blowoff ports.

Accordingly, the active ingredient can be blown out in different directions, and therefore, the active ingredient can be more efficiently supplied to the interior of the vehicle.

Moreover, in the active ingredient generation device according to an aspect of the present disclosure, the first flow channel may narrow toward the first blowoff port in an up-down direction of the vehicle in a state where the active ingredient generation device is attached to the ceiling.

Accordingly, the first air stream can be more efficiently blown out from the first blowoff port, and the active ingredient can be more efficiently drawn by the first air stream, and therefore, the active ingredient can be more efficiently supplied to the interior of the vehicle.

Moreover, in the active ingredient generation device according to an aspect of the present disclosure, the active ingredient generator includes a second air blower that generates a second air stream that flows through the second flow channel toward the second blowoff port, and a power of the second air blower may be lower than a power of the first air blower.

Accordingly, the active ingredient can be carried toward the second blowoff port by the second air stream while reducing damage to the active ingredient, and the active ingredient can be more easily drawn by the first air stream, and therefore, the active ingredient can be more efficiently supplied to the interior of the vehicle.

Moreover, the active ingredient generation device according to an aspect of the present disclosure may further include a light source that emits light for illuminating the interior of the vehicle.

Accordingly, the interior of the vehicle can be illuminated.

In the following, embodiments will be specifically described with reference to the drawings.

Note that the all embodiments described below show comprehensive or specific examples. The numerical values, shapes, materials, constituent elements, arrangements and connections of constituent elements, steps, orders of steps shown in the embodiments described below are examples and are not intended to limit the present disclosure. In addition, among the constituent elements in the embodiments described below, constituent elements that are not mentioned in the independent claims will be described as optional constituent elements.

Furthermore, the drawings are schematic diagrams and are not necessarily to the correct scale. Furthermore, throughout the drawings, the same constituent members are denoted by the same reference numerals.

### Embodiment 1

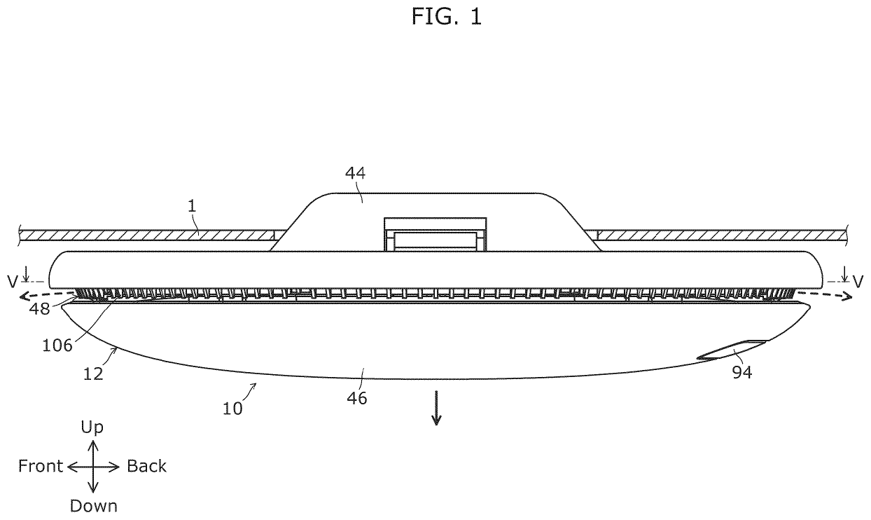

FIG. 1 is a side view of active ingredient generation device 10 according to Embodiment 1 and its surroundings. FIG. 1 illustrates a cross section of ceiling 1 of an interior of a vehicle. In the following, a front-back direction of the vehicle may be referred to simply as a front-back direction. Furthermore, a left-right direction of the vehicle may be referred to simply as a left-right direction. Furthermore, an up-down direction of the vehicle may be referred to simply as an up-down direction.

As illustrated in FIG. 1, active ingredient generation device 10 is attached to ceiling 1 of the interior of the vehicle. To be more specific, active ingredient generation device 10 is located inside the vehicle and attached to ceiling 1 of the vehicle. The vehicle is a car, a ship, or an aircraft, for example. The car is an automobile, for example. Active ingredient generation device 10 generates an active ingredient. Note that the “active ingredient” in the present disclosure means a charged particle solution containing OH radicals, OH radical, O2 radical, negative ion, positive ion, ozone, or nitrite ion, for example. These active ingredients serve as a base for achieving sanitization, deodorization, moisturizing, freshness retention, and virus inactivation as well as other useful effects in various situations

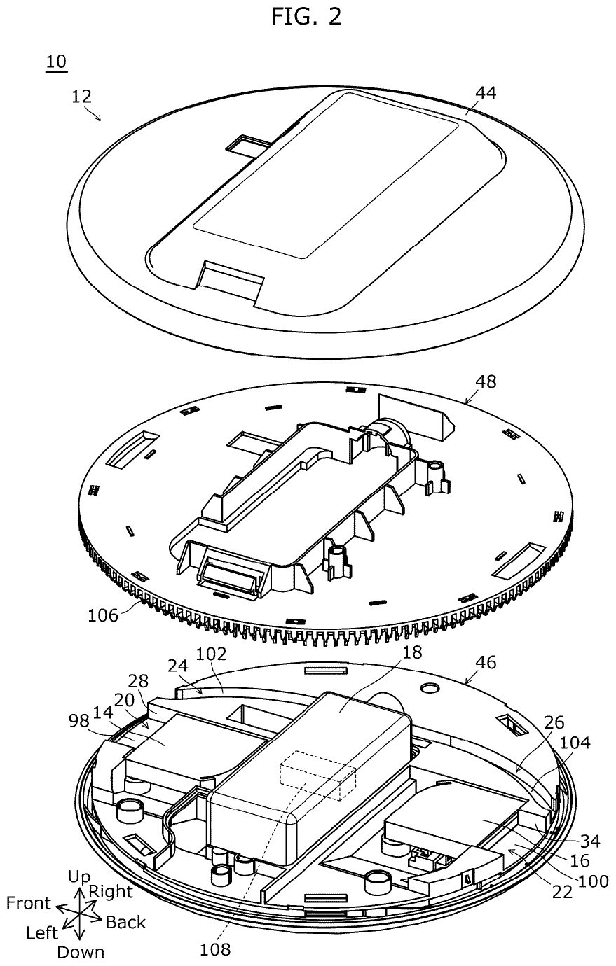

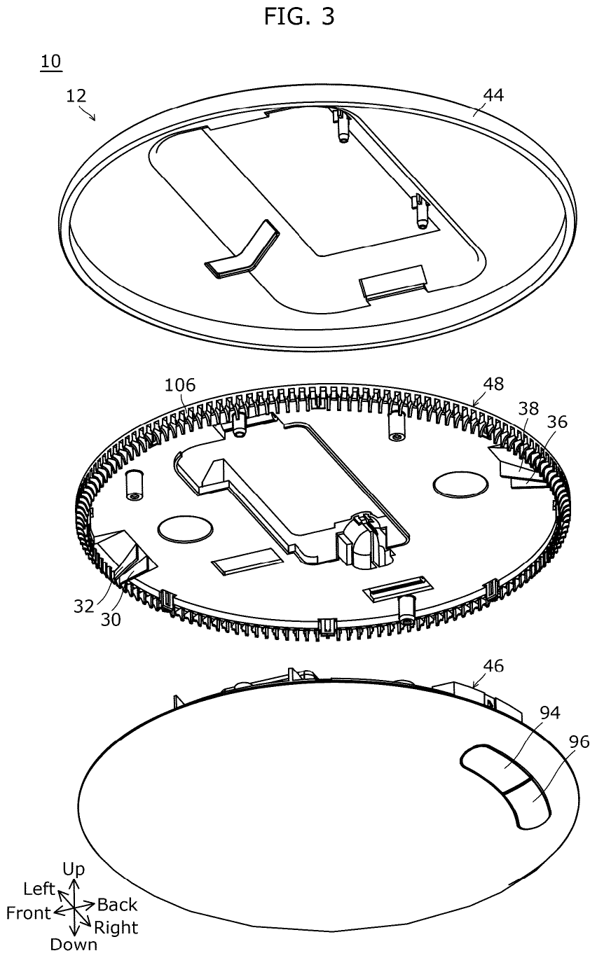

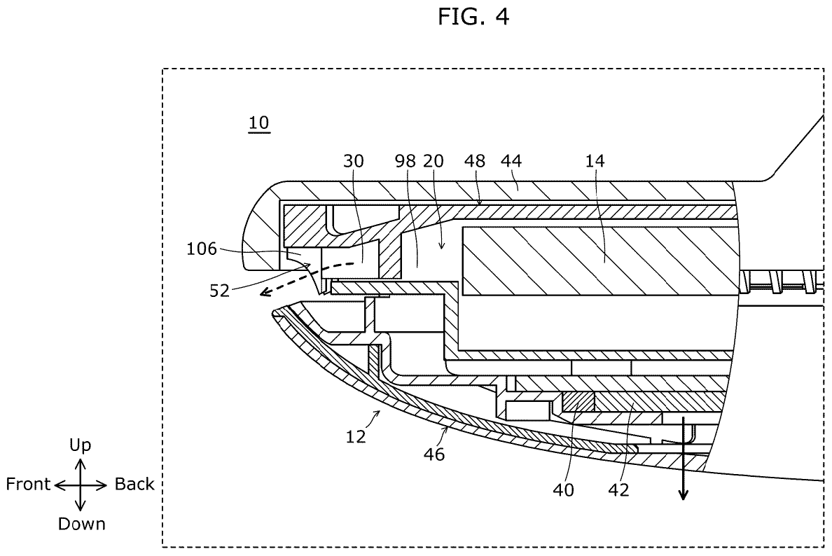

FIG. 2 is an exploded perspective view of active ingredient generation device 10 in FIG. 1 viewed from diagonally above. FIG. 3 is an exploded perspective view of active ingredient generation device 10 in FIG. 1 viewed from diagonally below. FIG. 4 is a partial cross-sectional view of active ingredient generation device 10 in FIG. 1. FIG. 4 illustrates a cross section that is perpendicular to the left-right direction and passes through wall portion 30.

As illustrated in FIGS. 1 to 4, active ingredient generation device 10 includes body 12, a plurality of first air blowers 14 and 16, active ingredient generator 18, a plurality of first flow channels 20 and 22, a plurality of second flow channels 24 and 26, a plurality of wall portions 28, 30, 32, 34, 36, and 38, light source 40, and light guide 42.

Body 12 includes attachment 44, body unit 46, cover portion 48, and a plurality of buttons 94 and 96.

Attachment 44 is attached to ceiling 1. For example, attachment 44 is attached to ceiling 1 with a bolt (not illustrated) or the like. Note that attachment 44 may be attached to ceiling 1 by being fitted to ceiling 1. Active ingredient generation device 10 is attached to ceiling 1 by attachment 44 being attached to ceiling 1. Attachment 44 is located above body unit 46 and cover portion 48 in the up-down direction in a state where active ingredient generation device 10 is attached to ceiling 1.

Body unit 46 is attached to cover portion 48. Body unit 46 is attached to cover portion 48 with a bolt or the like. Note that body unit 46 may be attached to cover portion 48 by being fitted to cover portion 48.

Body unit 46 includes a plurality of groove portions 98, 100, 102, and 104. Each of the groove portions 98, 100, 102, and 104 is recessed downward in the up-down direction in the state where active ingredient generation device 10 is attached to ceiling 1.

Body unit 46 houses light source 40 and light guide 42. Light source 40 is a light source that emits light to illuminate the interior of the vehicle. For example, light source 40 is an LED light source or the like. The lower surface of body unit 46 in the state where active ingredient generation device 10 is attached to ceiling 1 is light-transmissive. Light emitted from light source 40 is incident on light guide 42, is then emitted from light guide 42, and then exits from the lower surface of body unit 46 to illuminate the interior of the vehicle (see the thick solid arrows in FIGS. 1 to 4). Note that, for example, light may be emitted from light source 40 to ceiling 1 to indirectly illuminate the interior of the vehicle. Furthermore, active ingredient generation device 10 may include a plurality of light sources 40.

Cover portion 48 is attached to attachment 44. Cover portion 48 is attached to attachment 44 by being fitted to attachment 44. Note that cover portion 48 may be attached to attachment 44 with a bolt (not illustrated) or the like. Cover portion 48 is located above body unit 46 in the up-down direction in the state where active ingredient generation device 10 is attached to ceiling 1. Cover portion 48 includes a plurality of protrusions 106.

The plurality of protrusions 106 are annularly arranged around the up-down direction so as to surround the plurality of groove portions 98, 100, 102, and 104 in the state where active ingredient generation device 10 is attached to ceiling 1. More specifically, the plurality of protrusions 106 are annularly arranged in the circumferential direction when viewed in the up-down direction, so as to surround the plurality of groove portions 98, 100, 102, and 104 in the state where active ingredient generation device 10 is attached to ceiling 1. Each of the plurality of protrusions 106 protrudes downward in the up-down direction in the state where active ingredient generation device 10 is attached to ceiling 1.

Button 94 is a button for powering on and off the plurality of first air blowers 14 and 16 and active ingredient generator 18. For example, when button 94 is pressed in a state where the plurality of first air blowers 14 and 16 and active ingredient generator 18 are powered off, the plurality of first air blowers 14 and 16 and active ingredient generator 18 are powered on, and active ingredient generation device 10 supplies an active ingredient to the interior of the vehicle. When button 94 is pressed in a state where the plurality of first air blowers 14 and 16 and active ingredient generator 18 are powered on, the plurality of first air blowers 14 and 16 and active ingredient generator 18 are powered off, and active ingredient generation device 10 stop supplying the active ingredient to the interior of the vehicle.

Button 96 is a button for powering on and off light source 40. For example, when button 96 is pressed in a state where light source 40 is powered off, light source 40 is powered on, and active ingredient generation device 10 illuminates the interior of the vehicle. When button 96 is pressed in a state where light source 40 is powered on, light source 40 is powered off, and active ingredient generation device 10 stop illuminating the interior of the vehicle.

Each of the first air blowers 14 and 16 is provided inside body 12, and generates a first air stream. In the present embodiment, each of the plurality of first air blowers 14 and 16 is provided between body unit 46 and cover portion 48.

Active ingredient generator 18 is provided inside body 12, and generates an active ingredient. In the present embodiment, a discharge port for discharging the active ingredient in active ingredient generator 18 to the outside is provided between body unit 46 and cover portion 48. For example, active ingredient generator 18 generates the active ingredient by applying a high voltage to moisture in the air. Active ingredient generator 18 includes second air blower 108.

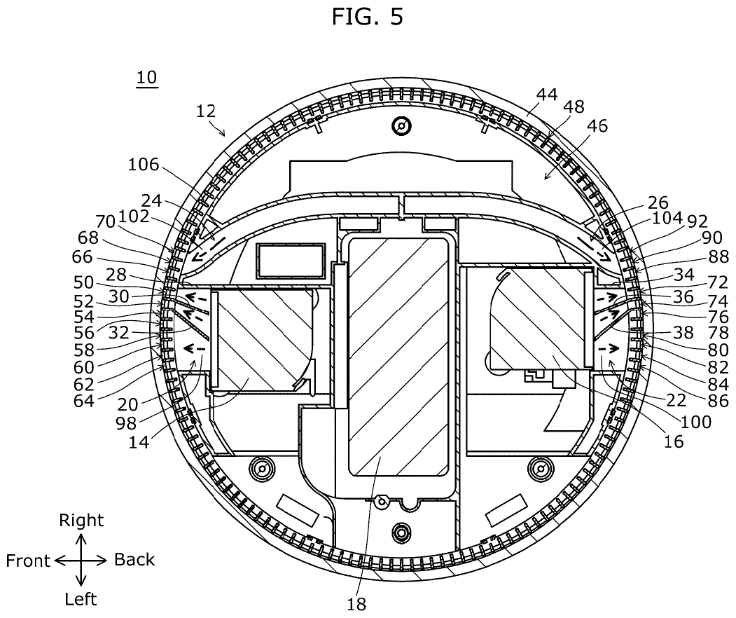

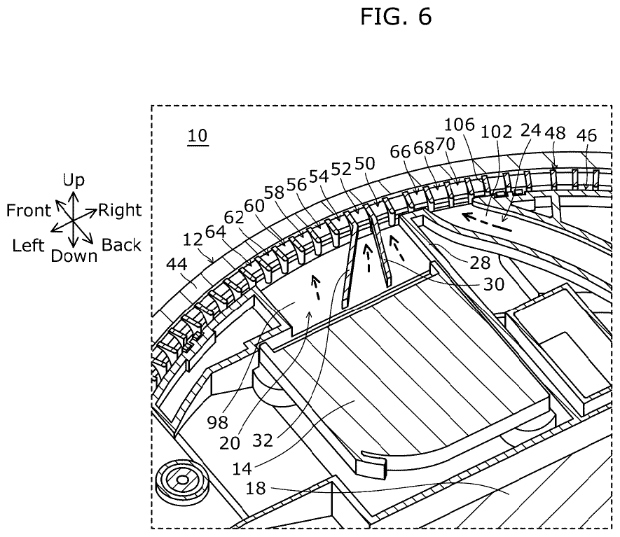

Second air blower 108 generates a second air stream that flows in second flow channel 24 toward second blowoff port 66 (68, 70) (see FIGS. 5 and 6). Furthermore, second air blower 108 generates a second air stream that flows in second flow channel 26 toward second blowoff port 88 (90, 92) (see FIGS. 5 and 6). The power of second air blower 108 is lower than the power of each of the plurality of first air blowers 14 and 16. First flow channel 20 is a flow channel formed inside body 12 through which the first air stream generated by first air blower 14 flows. In the present embodiment, first flow channel 20 is formed by groove portion 98, cover portion 48 and the like. The first air stream generated by first air blower 14 flows frontward in the front-back direction in the state where active ingredient generation device 10 is attached to ceiling 1.

First flow channel 20 is formed to become narrower toward first blowoff port 50 (52 to 64) (see FIGS. 5 and 6) in the up-down direction in the state where active ingredient generation device 10 is attached to ceiling 1. In the present embodiment, first flow channel 20 is formed to become gradually narrower toward first blowoff port 50 (52 to 64) by cover portion 48 being inclined downward toward first blowoff port 50 (52 to 64) in the state where active ingredient generation device 10 is attached to ceiling 1.

First flow channel 22 is a flow channel formed inside body 12 through which the first air stream generated by first air blower 16 flows. In the present embodiment, first flow channel 22 is formed by groove portion 100, cover portion 48 and the like. First flow channel 22 is located further back than first flow channel 20 in the state where active ingredient generation device 10 is attached to ceiling 1. The first air stream generated by first air blower 16 flows backward in the front-back direction in the state where active ingredient generation device 10 is attached to ceiling 1.

First flow channel 22 is formed to become narrower toward first blowoff port 72 (74 to 86) (see FIG. 5) in the up-down direction in the state where active ingredient generation device 10 is attached to ceiling 1. In the present embodiment, first flow channel 22 is formed to become gradually narrower toward first blowoff port 72 (74 to 86) by cover portion 48 being inclined downward toward first blowoff port 72 (74 to 86) in the state where active ingredient generation device 10 is attached to ceiling 1.

Each of the plurality of second flow channels 24 and 26 is a flow channel through which the active ingredient generated by active ingredient generator 18 flows, and is disposed inside body 12. In the present embodiment, second flow channel 24 is formed by groove portion 102, cover portion 48 and the like. Second flow channel 26 is formed by groove portion 104, cover portion 48, and the like. In the state where active ingredient generation device 10 is attached to ceiling 1, the active ingredient generated by active ingredient generator 18 flows in second flow channel 24 frontward in the front-back direction and flows in second flow channel 26 backward in the front-back direction.

FIG. 5 is a cross-sectional view of active ingredient generation device 10 taken along line V-V in FIG. 1. FIG. 6 is a perspective cross-sectional view of active ingredient generation device 10 taken along line V-V in FIG. 1. In FIGS. 5 and 6, illustration of the internal structures of the plurality of first air blowers 14 and 16 and active ingredient generator 18 are omitted.

Body 12 further includes a plurality of first blowoff ports 50, 52, 54, 56, 58, 60, 62, and 64, a plurality of second blowoff ports 66, 68, and 70, a plurality of first blowoff ports 72, 74, 76, 78, 80, 82, 84, and 86, and a plurality of second blowoff ports 88, 90, and 92.

The first air stream flowing through first flow channel 20 is blown out from each of the plurality of first blowoff ports 50 to 64 that are in communication with first flow channel 20 and the outside of body 12. In the present embodiment, each of the plurality of first blowoff ports 50 to 64 is a space between two adjacent protrusions 106 among the plurality of protrusions 106.

The width of each of the plurality of first blowoff ports 50 to 64 is smaller than the width of first flow channel 20. For example, the width of each of the plurality of first blowoff ports 50 to 64 and the width of first flow channel 20 are dimensions in a direction that is perpendicular to the direction in which the first air stream flows and perpendicular to the up-down direction in the state where active ingredient generation device 10 is attached to ceiling 1.

The plurality of first blowoff ports 50 to 64 are arranged in a direction around the up-down direction in the state where active ingredient generation device 10 is attached to ceiling 1. That is, the plurality of first blowoff ports 50 to 64 are arranged in the circumferential direction when viewed in the up-down direction in the state where active ingredient generation device 10 is attached to ceiling 1.

Inside body 12, a pair of wall portions 28 and 30 that guide the first air stream to one of the plurality of first blowoff ports 50 to 64 are formed. The pair of wall portions 28 and 30 guide the first air stream to first blowoff port 50. The pair of wall portions 28 and 30 are formed in such a manner that the distance between the pair of wall portions 28 and 30 decreases toward the one (first blowoff port 50) of the plurality of first blowoff ports 50 to 64. The one (first blowoff port 50) of the plurality of first blowoff ports 50 to 64 is the closest to second blowoff port 66 (68, 70) among the plurality of first blowoff ports 50 to 64.

Inside body 12, a pair of wall portions 30 and 32 that guide the first air stream to one of the plurality of first blowoff ports 50 to 64 are formed. The pair of wall portions 30 and 32 guide the first air stream to first blowoff port 52. The pair of wall portions 30 and 32 are formed in such a manner that the distance between the pair of wall portions 30 and 32 decreases toward the one (first blowoff port 52) of the plurality of first blowoff ports 50 to 64.

The active ingredient flowing through second flow channel 24 is blown out from each of the plurality of second blowoff ports 66 to 70 that are in communication with second flow channel 24 and the outside of body 12. In the present embodiment, each of the plurality of second blowoff ports 66 to 70 is a space between two adjacent protrusions 106 among the plurality of protrusions 106.

The plurality of second blowoff ports 66 to 70 are provided at a location where the active ingredient blown out from the plurality of second blowoff ports 66 to 70 is drawn by the first air stream blown out from first blowoff port 50 (52 to 64). That is, the active ingredient blown out from each of the plurality of second blowoff ports 66 to 70 is drawn by the first air stream blown out from first blowoff port 50 (52 to 64).

The plurality of second blowoff ports 66 to 70 may be provided at a location where the active ingredient flowing through second flow channel 24 is blown out from the plurality of second blowoff ports 66 to 70 by being drawn by the first air stream blown out from first blowoff port 50 (52 to 64). That is, the active ingredient flowing through second flow channel 24 may be blown out from second blowoff port 66 (68, 70) by being drawn by the first air stream blown out from first blowoff port 50 (52 to 64). Accordingly, second air blower 108 with lower power can be used. In the present embodiment, the plurality of second blowoff ports 66 to 70 are provided at the same height as the plurality of first blowoff ports 50 to 64 in the up-down direction in the state where active ingredient generation device 10 is attached to ceiling 1. In addition, the plurality of second blowoff ports 66 to 70 are provided to be adjacent to the plurality of first blowoff ports 50 to 64 in the direction around the up-down direction in the state where active ingredient generation device 10 is attached to ceiling 1. That is, the plurality of second blowoff ports 66 to 70 are provided to be adjacent to the plurality of first blowoff ports 50 to 64 in the circumferential direction when viewed in the up-down direction in the state where active ingredient generation device 10 is attached to ceiling 1.

The plurality of second blowoff ports 66 to 70 are arranged in the direction around the up-down direction in the state where active ingredient generation device 10 is attached to ceiling 1. That is, the plurality of second blowoff ports 66 to 70 are arranged in the circumferential direction when viewed in the up-down direction in the state where active ingredient generation device 10 is attached to ceiling 1.

The flow velocity of the first air stream blown out from first blowoff port 50 (52 to 64) is higher than the flow velocity of the active ingredient blown out from each of the plurality of second blowoff ports 66 to 70.

The first air stream flowing through first flow channel 22 is blown out from each of the plurality of first blowoff ports 72 to 86 that are in communication with first flow channel 22 and the outside of body 12. In the present embodiment, each of the plurality of first blowoff ports 72 to 86 is a space between two adjacent protrusions 106 among the plurality of protrusions 106.

The width of each of the plurality of first blowoff ports 72 to 86 is smaller than the width of first flow channel 22. For example, the width of each of the plurality of first blowoff ports 72 to 86 and the width of first flow channel 22 are dimensions in a direction that is perpendicular to the direction in which the first air stream flows and perpendicular to the up-down direction in the state where active ingredient generation device 10 is attached to ceiling 1.

The plurality of first blowoff ports 72 to 86 are arranged in the direction around the up-down direction in the state where active ingredient generation device 10 is attached to ceiling 1. That is, the plurality of first blowoff ports 72 to 86 are arranged in the circumferential direction when viewed in the up-down direction in the state where active ingredient generation device 10 is attached to ceiling 1.

Inside body 12, a pair of wall portions 34 and 36 that guide the first air stream to one of the plurality of first blowoff ports 72 to 86 are formed. The pair of wall portions 34 and 36 guide the first air stream to first blowoff port 72. The pair of wall portions 34 and 36 are formed in such a manner that the distance between the pair of wall portions 34 and 36 decreases toward the one (first blowoff port 72) of the plurality of first blowoff ports 72 to 86. The one (first blowoff port 72) of the plurality of first blowoff ports 72 to 86 is the closest to second blowoff port 88 (90, 92) among the plurality of first blowoff ports 72 to 86.

Inside body 12, a pair of wall portions 36 and 38 that guide the first air stream to one of the plurality of first blowoff ports 72 to 86 are formed. The pair of wall portions 36 and 38 guide the first air stream to first blowoff port 74. The pair of wall portions 36 and 38 are formed in such a manner that the distance between the pair of wall portions 36 and 38 decreases toward the one (first blowoff port 74) of the plurality of first blowoff ports 74 to 86.

The active ingredient flowing through second flow channel 26 is blown out from each of the plurality of second blowoff ports 88 to 92 that are in communication with second flow channel 26 and the outside of body 12. In the present embodiment, each of the plurality of second blowoff ports 88 to 92 is a space between two adjacent protrusions 106 among the plurality of protrusions 106.

The plurality of second blowoff ports 88 to 92 are provided at a location where the active ingredient blown out from the second blowoff ports 88 to 92 are drawn by the first air stream blown out from first blowoff port 72 (74 to 86). That is, the active ingredient blown out from each of the plurality of second blowoff ports 88 to 92 is drawn by the first air stream blown out from first blowoff port 72 (74 to 86).

The plurality of second blowoff ports 88 to 92 may be provided at a location where the active ingredient flowing through second flow channel 26 is blown out from the plurality of second blowoff ports 88 to 92 by being drawn by the first air stream blown out from first blowoff port 72 (74 to 86). That is, the active ingredient flowing through second flow channel 26 may be blown out from second blowoff port 88 (90, 92) by being drawn by the first air stream blown out from first blowoff port 72 (74 to 86). Accordingly, second air blower 108 with lower power can be used. In the present embodiment, the plurality of second blowoff ports 88 to 92 are provided at the same height as the plurality of first blowoff ports 72 to 86 in the up-down direction in the state where active ingredient generation device 10 is attached to ceiling 1. In addition, the plurality of second blowoff ports 88 to 92 are provided to be adjacent to the plurality of first blowoff ports 72 to 86 in the direction around the up-down direction in the state where active ingredient generation device 10 is attached to ceiling 1. That is, the plurality of second blowoff ports 88 to 92 are provided to be adjacent to the plurality of first blowoff ports 72 to 86 in the circumferential direction when viewed in the up-down direction in the state where active ingredient generation device 10 is attached to ceiling 1.

The plurality of second blowoff ports 88 to 92 are arranged in the direction around the up-down direction in the state where active ingredient generation device 10 is attached to ceiling 1. That is, the plurality of second blowoff ports 88 to 92 are arranged in the circumferential direction when viewed in the up-down direction in the state where active ingredient generation device 10 is attached to ceiling 1.

The flow velocity of the first air stream blown out from first blowoff port 72 (74 to 86) is higher than the flow velocity of the active ingredient blown out from each of the plurality of second blowoff ports 88 to 92.

The first air streams flowing through first flow channels 20 and 22 are blown out from first blowoff ports 50 (52 to 64) and 72 (74 to 86) that are in communication with first flow channels 20 and 22, respectively, and with the outside of body 12. More specifically, the first air stream flowing through first flow channel 20 is blown out from first blowoff port 50 (52 to 64) that is in communication with first flow channel 20 and the outside of body 12. Moreover, the first air stream flowing through first flow channel 22 is blown out from first blowoff port 72 (74 to 86) that is in communication with first flow channel 22 and the outside of body 12.

The active ingredient flowing second flow channels 24 and 26 are blown out from second blowoff ports 66 (68, 70) and 88 (90, 92) that are in communication with second flow channels 24 and 26, respectively, and with the outside of body 12. More specifically, the second air stream flowing through second flow channel 24 is blown out from second blowoff port 66 (68, 70) that is in communication with second flow channel 24 and the outside of body 12. Moreover, the second air stream flowing through second flow channel 24 is blown out from second blowoff port 88 (90, 92) that is in communication with second flow channel 24 and the outside of body 12.

Second blowoff ports 66 (68, 70) and 88 (90, 92) are provided at locations where the active ingredient flowing through the second flow channels 24 and 26 that are in communication with second blowoff ports 66 (68, 70) and 88 (90, 92) is blown out from second blowoff ports 66 (68, 70) and 88 (90, 92) by being drawn by the first air streams blown out from first blowoff ports 50 (52 to 64) and 72 (74 to 86). More specifically, second blowoff port 66 (68, 70) is provided at a location where the active ingredient flowing through second flow channel 24 that is in communication with second blowoff port 66 (68, 70) is blown out from second blowoff port 66 (68, 70) by being drawn by the first air stream blown out from first blowoff port 50 (52 to 64). Second blowoff port 88 (90, 92) is provided at a location where the active ingredient flowing through second flow channel 26 that is in communication with second blowoff port 88 (90, 92) is blown out from second blowoff port 88 (90, 92) by being drawn by the first air stream blown out from first blowoff port 72 (74 to 86).

In active ingredient generation device 10, the active ingredient flowing through second flow channel 24 is drawn by the first air stream (see the thick dashed line arrows in FIGS. 1, 4, 5, and 6) blown out from first blowoff port 50 (52 to 64) (see the thick alternate long and short dashed line arrows in FIGS. 5 and 6), and is blown out from second blowoff port 66 (68, 70).

The active ingredient flowing through second flow channel 26 is drawn by the first air stream (see the thick dashed line arrows in FIGS. 1 and 5) blown out from first blowoff port 72 (74 to 86) (see the thick alternate long and short dashed line arrow in FIG. 5), and is blown out from second blowoff port 88 (90, 92).

Active ingredient generation device 10 according to the present embodiment is an active ingredient generation device that is attached to ceiling 1 of the interior of a vehicle, and includes: body 12; first air blower 14 that is provided inside body 12 and generates a first air stream; active ingredient generator 18 that is provided inside body 12 and generates an active ingredient; first flow channel 20 through which the first air stream generated by first air blower 14 flows, first flow channel 20 being disposed inside body 12; and second flow channel 24 through which the active ingredient generated by active ingredient generator 18 flows, second flow channel 24 being disposed inside body 12. Body 12 includes: first blowoff port 50 (52 to 64) from which he first air stream flowing through first flow channel 20 is blown out, first blowoff port 50 (52 to 64) being in communication with first flow channel 20 and the outside of body 12; and second blowoff port 66 (68, 70) from which the active ingredient flowing through second flow channel 24 is blown out, second blowoff port 66 (68, 70) being in communication with second flow channel 24 and the outside of body 12. Second blowoff port 66 (68, 70) is provided at a location where the active ingredient blown out from second blowoff port 66 (68, 70) is drawn by the first air stream blown out from first blowoff port 50 (52 to 64).

Accordingly, second blowoff port 66 (68, 70) is provided at a location where the active ingredient blown out from second blowoff port 66 (68, 70) is drawn by the first air stream blown out from first blowoff port 50 (52 to 64). Therefore, the active ingredient blown out from second blowoff port 66 (68, 70) can be drawn by the first air stream blown out from first blowoff port 50 (52 to 64) even if first flow channel 20 and second flow channel 24 are not merged with each other in body 12. Therefore, the active ingredient can be efficiently supplied to the interior of the vehicle by the action of the first air stream while avoiding the complication or the like of the structure arising from merging of first flow channel 20 and second flow channel 24 in body 12.

Furthermore, in active ingredient generation device 10 according to the present embodiment, second blowoff port 66 (68, 70) is provided at a location where the active ingredient flowing through second flow channel 24 is blown out from second blowoff port 66 (68, 70) by being drawn by the first air stream blown out from first blowoff port 50 (52 to 64).

Accordingly, second blowoff port 66 (68, 70) is provided at a location where the active ingredient flowing through second flow channel 24 is blown out from second blowoff port 66 (68, 70) by being drawn by the first air stream blown out from first blowoff port 50 (52 to 64). Therefore, even if first flow channel 20 and second flow channel 24 are not merged with each other in body 12, the active ingredient flowing through second flow channel 24 can be blown out from second blowoff port 66 (68, 70) by the action of the first air stream. Therefore, the active ingredient can be more efficiently supplied to the interior of the vehicle while avoiding the complication or the like of the structure arising from merging of first flow channel 20 and second flow channel 24 in body 12.

Furthermore, in active ingredient generation device 10 according to the present embodiment, the flow velocity of the first air stream blown out from first blowoff port 50 (52 to 64) is higher than the flow velocity of the active ingredient blown out from second blowoff port 66 (68, 70).

Accordingly, the active ingredient is more easily drawn by the first air stream, and therefore, the active ingredient can be more efficiently supplied to the interior of the vehicle. Furthermore, in active ingredient generation device 10 according to the present embodiment, the width of first blowoff port 50 (52 to 64) is smaller than the width of first flow channel 20.

Accordingly, the flow velocity of the first air stream blown out from first blowoff port 50 (52 to 64) can be increased, and the active ingredient is more easily drawn by the first air stream, and therefore, the active ingredient can be more efficiently supplied to the interior of the vehicle.

Furthermore, in active ingredient generation device 10 according to the present embodiment, body 12 includes a plurality of first blowoff ports 50 to 64, a pair of wall portions 28 and 30 that guide the first air stream to one (first blowoff port 50) of the plurality of first blowoff ports 50 to 64 are formed inside body 12, and the pair of wall portions 28 and 30 are formed in such a manner that the distance between the pair of wall portions 28 and 30 decreases toward the one (first blowoff port 50) of the plurality of first blowoff ports 50 to 64.

Accordingly, the first air stream can be more efficiently blown out from first blowoff port 50, and the active ingredient can be more efficiently drawn by the first air stream, and therefore, the active ingredient can be more efficiently supplied to the interior of the vehicle.

Furthermore, in active ingredient generation device 10 according to the present embodiment, the one (first blowoff port 50) of the plurality of first blowoff ports 50 to 64 is the closest to second blowoff port 66 (68, 70) among the plurality of first blowoff ports 50 to 64.

Accordingly, the first air stream can be more efficiently blown out from first blowoff port 50, which is the closest to second blowoff port 66 (68, 70) among the plurality of first blowoff ports 50 to 64, and the active ingredient can be more efficiently drawn by the first air stream, and therefore, the active ingredient can be more efficiently supplied to the interior of the vehicle.

Furthermore, active ingredient generation device 10 according to the present embodiment includes a plurality of first flow channels 20 and 22 and a plurality of second flow channels 24 and 26. Body 12 includes: a plurality of first blowoff ports 50 (52 to 64) and 72 (74 to 86) from which first air streams flowing through the plurality of first flow channels 20 and 22 are blown out, the plurality of first blowoff ports 50 (52 to 64) and 72 (74 to 86) being in communication with the plurality of first flow channels 20 and 22 and the outside of body 12; and a plurality of second blowoff ports 66 (68, 70) and 88 (90, 92) from which the active ingredient flowing through the plurality of second flow channels 24 and 26 are blown out, the plurality of second blowoff ports 66 (68, 70) and 88 (90, 92) being in communication with the plurality of second flow channels 24 and 26 and the outside of body 12. The plurality of second blowoff ports 66 (68, 70) and 88 (90, 92) are provided at locations where the active ingredient flowing through the plurality of second flow channels 24 and 26 that are in communication with the plurality of second blowoff ports 66 (68, 70) and 88 (90, 92) is blown out from the plurality of second blowoff ports 66 (68, 70) and 88 (90, 92) by being drawn by the first air streams blown out from the plurality of first blowoff ports 50 (52 to 64) and 72 (74 to 86).

Accordingly, the active ingredient can be blown out in different directions, and therefore, the active ingredient can be more efficiently supplied to the interior of the vehicle.

Furthermore, in active ingredient generation device 10 according to the present embodiment, first flow channel 20 is formed to become narrower toward first blowoff port 50 (52 to 64) in the up-down direction of the vehicle in the state where active ingredient generation device 10 is attached to ceiling 1.

Accordingly, the first air stream can be more efficiently blown out from first blowoff port 50 (52 to 64), and the active ingredient can be more efficiently drawn by the first air stream, and therefore, the active ingredient can be more efficiently supplied to the interior of the vehicle.

Furthermore, in active ingredient generation device 10 according to the present embodiment, active ingredient generator 18 includes second air blower 108 that generates a second air stream that flows through second flow channel 24 toward second blowoff port 66 (68, 70), and the power of second air blower 108 is lower than the power of first air blower 14.

Accordingly, the active ingredient can be carried toward second blowoff port 66 (68, 70) by the second air stream while reducing damage to the active ingredient, and the active ingredient can be more easily drawn by the first air stream, and therefore, the active ingredient can be more efficiently supplied to the interior of the vehicle.

Furthermore, active ingredient generation device 10 according to the present embodiment includes a light source that emits light that illuminates the interior of the vehicle.

Accordingly, the interior of the vehicle can be illuminated.

### Other Embodiments

Although an active ingredient generation device according to one or more aspects has been described above with reference to an embodiment, the present disclosure is not limited to this embodiment. Various modifications to the embodiment that occur to those skilled in the art may be included in the scope of the present disclosure without departing from the spirit of the present disclosure.

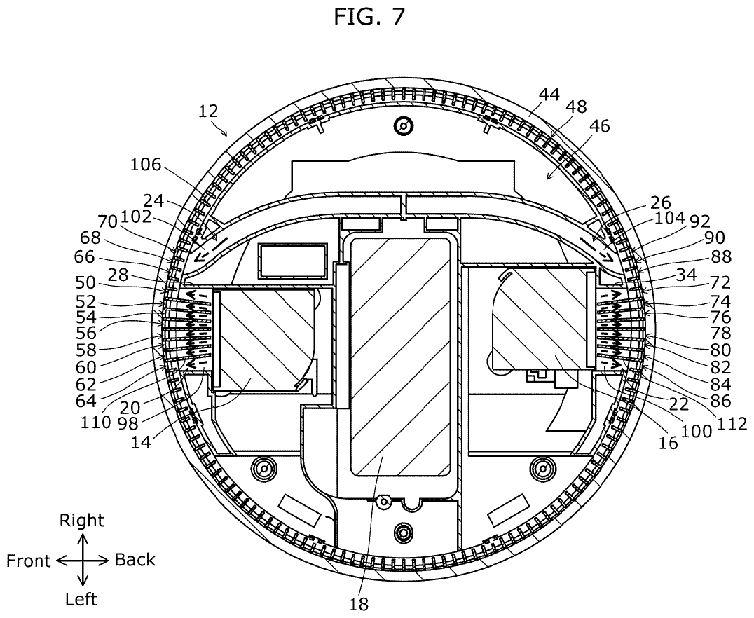

Although a case where a plurality of wall portions 30, 32, 36, and 38 are formed has been described in the above embodiment, the present disclosure is not limited to this case. FIG. 7 is a cross-sectional view illustrating an active ingredient generation device according to Embodiment 2. As illustrated in FIG. 7, for example, as an alternative to the plurality of wall portions 30 and 32, a plurality of wall portions 110 may be formed. In addition, as an alternative to the plurality of wall portions 36 and 38, a plurality of wall portions 112 may be formed.

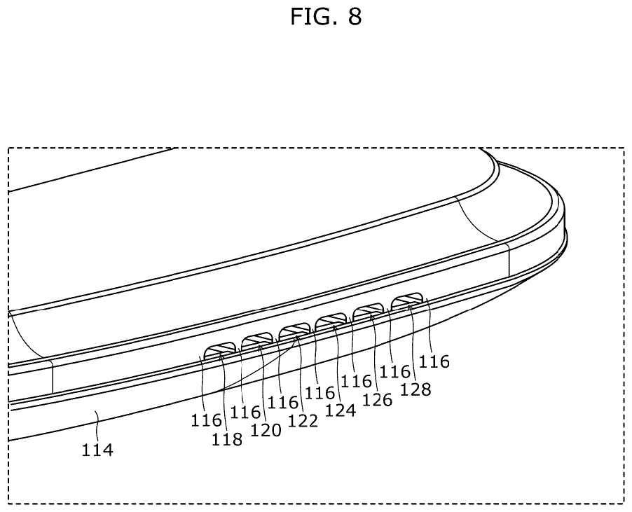

Furthermore, although a case where body unit 46 and the plurality of protrusions 106 are spaced apart from each other (see FIG. 4) has been described in the above embodiment, the present disclosure is not limited to this case. FIG. 8 is a perspective view illustrating a part of an active ingredient generation device according to Embodiment 3. As illustrated in FIG. 8, for example, body unit 114 and a plurality of protrusions 116 may be in contact with each other. In this case, each of a plurality of first blowoff ports 118, 120, 122, 124, and 126 is formed by adjacent two protrusions 116 and body unit 114. The active ingredient flowing through the second flow channel can be blown out from second blowoff port 128 by the action of the first air stream blown out from the plurality of first blowoff ports 118 to 126.

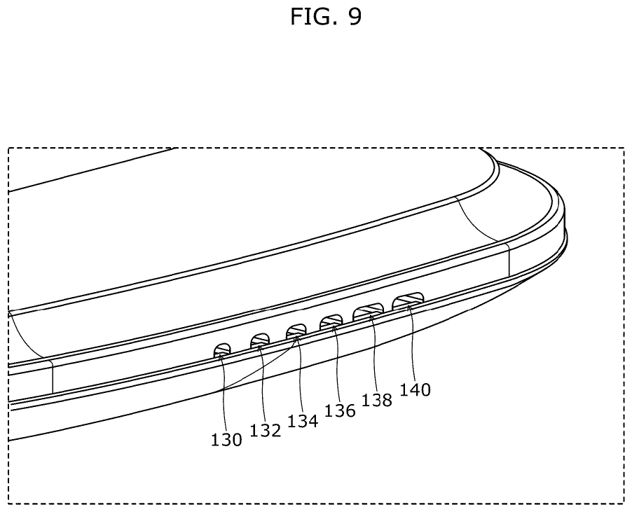

Furthermore, although a case where the plurality of first blowoff ports 50 to 64 have the same width has been described in the above embodiment, the present disclosure is not limited to this case. FIG. 9 is a perspective view illustrating a part of an active ingredient generation device according to Embodiment 4. As illustrated in FIG. 9, for example, a plurality of first blowoff ports 130, 132, 134, 136, and 138 may have different widths. The narrower the width of the first blowoff port, the higher the flow velocity of the first air stream blown out therefrom is. Therefore, by varying the width of the plurality of first blowoff ports 130 to 138, the first air stream can be blown out in a direction different from the direction in which the plurality of first blowoff ports 130 to 138 open. Therefore, the active ingredient flowing through the second flow channel can be blown out from second blowoff port 140 in a direction different from the direction in which the plurality of first blowoff ports 130 to 138 open.

In the active ingredient generation device according to the present embodiment, the body includes a plurality of first blowoff ports 130 to 138, and the plurality of first blowoff ports 130 to 138 have different widths.

Accordingly, the first air stream can be blown out from the plurality of first blowoff ports 130 to 138 at different flow velocities, and the first air stream can be blown out in a desired direction. Therefore, the active ingredient can be blown out in a desired direction and can be more efficiently supplied to the interior of the vehicle.

Furthermore, although a case where the plurality of second blowoff ports 66 to 70 (88 to 92) are provided to be adjacent to the plurality of first blowoff ports 50 to 64 (72 to 86) in the direction around the up-down direction in the state where active ingredient generation device 10 is attached to ceiling 1 has been described in the above embodiment, the present disclosure is not limited to this case. For example, a second blowoff port may be adjacent to a first blowoff port in the up-down direction in the state where the active ingredient generation device is attached to the ceiling. Alternatively, a second blowoff port needs not be adjacent to a first blowoff port, as far as the second blowoff port is provided at a location where the active ingredient blown out from the second blowoff port is drawn by the first air stream blown out from the first blowoff port. Moreover, a second blowoff port may be provided at a location where the active ingredient flowing through the second flow channel is blown out from the second blowoff port by being drawn by a first air stream.

Furthermore, although a case where the width of each of the plurality of first blowoff ports 50 to 64 (72 to 86) is smaller than the width of first flow channel 20 (22) has been described in the above embodiment, the present disclosure is not limited to this case. For example, the width of the first blowoff ports may be the same as the width of the first flow channel or may be greater than the width of the first flow channel.

Furthermore, although a case where active ingredient generation device 10 includes the plurality of wall portions 30, 32, 36, and 38 has been described in the above embodiment, the present disclosure is not limited to this case. For example, active ingredient generation device 10 need not include the plurality of wall portions 30, 32, 36, and 38.

Furthermore, although a case where body 12 includes the plurality of first blowoff ports 50 to 64 (72 to 86) has been described in the above embodiment, the present disclosure is not limited to this case. For example, the body may include one first blowoff port for one first flow channel.

Furthermore, although a case where body 12 includes the plurality of second blowoff ports 66 to 70 (88 to 92) has been described in the above embodiment, the present disclosure is not limited to this case. For example, the body may include one second blowoff port for one second flow channel.

Furthermore, although a case where active ingredient generation device 10 includes the plurality of first air blowers 14 and 16, the plurality of first flow channels 20 and 22, and the plurality of second flow channels 24 and 26 has been described in the above embodiment, the present disclosure is not limited to this case. For example, the active ingredient generation device may include one first air blower, one first flow channel, and one second flow channel, rather than a plurality of first air blowers, a plurality of first flow channels, and a plurality of second flow channels.

Furthermore, although a case where first flow channel 20 is formed to become narrower toward first blowoff port 50 (52 to 64) in the up-down direction of the vehicle in the state where active ingredient generation device 10 is attached to ceiling 1 has been described in the above embodiment, the present disclosure is not limited to this case. The same holds true for first flow channel 22. For example, the first flow channel may be formed not to become narrower toward the first blowoff port in the up-down direction of the vehicle in the state where the active ingredient generation device is attached to the ceiling.

Furthermore, although a case where active ingredient generator 18 includes second air blower 108 that generates a second air stream that flows through second flow channel 24 toward second blowoff port 66 (68, 70) and generates a second air stream that flows through second flow channel 26 toward second blowoff port 88 (90, 92) has been described in the above embodiment, the present disclosure is not limited to this case. For example, active ingredient generator 18 need not include the second air blower. In this case, for example, the active ingredient flowing through second flow channel 24 is blown out from second blowoff port 66 (68, 70) by being drawn by the first air stream blown out from first blowoff port 50 (52 to 64), and the active ingredient flowing through second flow channel 26 is blown out from second blowoff port 88 (90, 92) by being drawn by the first air stream blown out from first blowoff port 72 (74 to 86).

Furthermore, although a case where active ingredient generation device 10 includes light source 40 has been described in the above embodiment, the present disclosure is not limited to this case. For example, active ingredient generation device 10 need not include light source 40.

Furthermore, although a case where active ingredient generation device 10 is attached to ceiling 1 of the interior of the vehicle in such an orientation that FIG. 1 is a view of active ingredient generation device 10 and its surroundings viewed from the left side in the left-right direction of the vehicle has been described in the above embodiment, the present disclosure is not limited to this case. For example, active ingredient generation device 10 may be attached to ceiling 1 of the interior of the vehicle in such an orientation that FIG. 1 is a view of active ingredient generation device 10 and its surroundings viewed from the front side or back side in the front-back direction of the vehicle.

While various embodiments have been described herein above, it is to be appreciated that various changes in form and detail may be made without departing from the spirit and scope of the present disclosure as presently or hereafter claimed.

### Notes

The above description of embodiments and the like discloses the following techniques.

### Technique 1

An active ingredient generation device that is attached to a ceiling of an interior of a vehicle includes: a body; a first air blower that is disposed inside the body and generates a first air stream; an active ingredient generator that is disposed inside the body and generates an active ingredient; a first flow channel through which the first air stream generated by the first air blower flows, the first flow channel being disposed inside the body; and a second flow channel through which the active ingredient generated by the active ingredient generator flows, the second flow channel being disposed inside the body. The body includes: a first blowoff port from which the first air stream flowing through the first flow channel is blown out, the first blowoff port being in communication with the first flow channel and an outside of the body; and a second blowoff port from which the active ingredient flowing through the second flow channel is blown out, the second blowoff port being in communication with the second flow channel and the outside of the body. The second blowoff port is disposed at a location where the active ingredient blown out from the second blowoff port is drawn by the first air stream blown out from the first blowoff port.

### Technique 2

In the active ingredient generation device according to Technique 1, the second blowoff port is disposed at a location where the active ingredient flowing through the second flow channel is blown out from the second blowoff port by being drawn by the first air stream blown out from the first blowoff port.

### Technique 3

In the active ingredient generation device according to Technique 1 or 2, a flow velocity of the first air stream blown out from the first blowoff port is higher than a flow velocity of the active ingredient blown out from the second blowoff port.

### Technique 4

In the active ingredient generation device according to any one of Techniques 1 to 3, a width of the first blowoff port is smaller than a width of the first flow channel.

### Technique 5

In the active ingredient generation device according to Technique 4, the body includes a plurality of first blowoff ports each being the first blowoff port, a pair of wall portions that guide the first air stream to one of the plurality of first blowoff ports are arranged inside the body, and a distance between the pair of wall portions decreases toward the one of the plurality of first blowoff ports.

### Technique 6

In the active ingredient generation device according to Technique 5, the one of the plurality of first blowoff ports is closest to the second blowoff port, among the plurality of first blowoff ports.

### Technique 7

In the active ingredient generation device according to Technique 4, the body includes a plurality of first blowoff ports each being the first blowoff port, and the plurality of first blowoff ports have different widths.

### Technique 8

In the active ingredient generation device according to any one of Techniques 1 to 4, the active ingredient generation device includes a plurality of first flow channels each being the first flow channel and a plurality of second flow channels each being the second flow channel. The body includes: a plurality of first blowoff ports from which first air streams flowing through the plurality of first flow channels are blown out, the plurality of first blowoff ports each being the first blowoff port, the plurality of first blowoff ports being in communication with the plurality of first flow channels and the outside of the body, the first air streams each being the first air stream; and a plurality of second blowoff ports from which the active ingredient flowing through the plurality of second flow channels is blown out, the plurality of second blowoff ports being in communication with the plurality of second flow channels and the outside of the body, the plurality of second blowoff ports each being the second blowoff port. The plurality of second blowoff ports are arranged at locations where the active ingredient flowing through the plurality of second flow channels that are in communication with the plurality of second blowoff ports is blown out from the plurality of second blowoff ports by being drawn by the first air streams blown out from the plurality of first blowoff ports.

### Technique 9

In the active ingredient generation device according to any one of Techniques 1 to 8, the first flow channel narrows toward the first blowoff port in an up-down direction of the vehicle in a state where the active ingredient generation device is attached to the ceiling.

### Technique 10

In the active ingredient generation device according to any one of Techniques 1 to 9, the active ingredient generator includes a second air blower that generates a second air stream that flows through the second flow channel toward the second blowoff port, and a power of the second air blower is lower than a power of the first air blower.

### Technique 11

The active ingredient generation device according to any one of Techniques 1 to 10 further includes a light source that emits light for illuminating the interior of the vehicle.

### Further Information about Technical Background to this Application

The disclosure of the following patent application including specification, drawings, and claims are incorporated herein y reference in their entirety: Japanese Patent Application No. 2022-194350 filed on Dec. 5, 2022 and PCT International Application No. PCT/JP2023/041593 filed on Nov. 20, 2023.

### INDUSTRIAL APPLICABILITY

The present disclosure is applicable to an active ingredient generation device provided in the interior of a vehicle.

1. An active ingredient generation device that is attached to a ceiling of an interior of a vehicle, the active ingredient generation device comprising:
a body;
a first air blower that is disposed inside the body and generates a first air stream;
an active ingredient generator that is disposed inside the body and generates an active ingredient;
a first flow channel through which the first air stream generated by the first air blower flows, the first flow channel being disposed inside the body; and
a second flow channel through which the active ingredient generated by the active ingredient generator flows, the second flow channel being disposed inside the body,
wherein the body includes:
a first blowoff port from which the first air stream flowing through the first flow channel is blown out, the first blowoff port being in communication with the first flow channel and an outside of the body; and
a second blowoff port from which the active ingredient flowing through the second flow channel is blown out, the second blowoff port being in communication with the second flow channel and the outside of the body,
the second blowoff port is disposed at a location where the active ingredient blown out from the second blowoff port is drawn by the first air stream blown out from the first blowoff port,
a width of the first blowoff port is smaller than a width of the first flow channel,
the body includes a plurality of first blowoff ports each being the first blowoff port,
a pair of wall portions that guide the first air stream to one of the plurality of first blowoff ports are arranged inside the body,
a distance between the pair of wall portions decreases toward the one of the plurality of first blowoff ports, and
the one of the plurality of first blowoff ports is closest to the second blowoff port, among the plurality of first blowoff ports.

2. The active ingredient generation device according to claim 1, wherein the second blowoff port is disposed at a location where the active ingredient flowing through the second flow channel is blown out from the second blowoff port by being drawn by the first air stream blown out from the first blowoff port.

3. The active ingredient generation device according to claim 1, wherein a flow velocity of the first air stream blown out from the first blowoff port is higher than a flow velocity of the active ingredient blown out from the second blowoff port.

4. The active ingredient generation device according to claim 1, wherein a width of the one of the plurality of first blowoff ports is smaller than a width of the second flow channel.

5. The active ingredient generation device according to claim 1, wherein the distance between the pair of wall portions that decreases toward the one of the plurality of first blowoff ports is smaller than a distance between a pair of wall portions that decreases toward an other one of the plurality of first blowoff ports, the other one of the plurality of first blowoff ports being disposed farther from the second blowoff port than the one of the plurality of first blowoff ports.

6. The active ingredient generation device according to claim 1, wherein the plurality of first blowoff ports have different widths.

7. The active ingredient generation device according to claim 1, wherein the active ingredient generation device comprises a plurality of first flow channels each being the first flow channel and a plurality of second flow channels each being the second flow channel,
the body includes:
the plurality of first blowoff ports from which first air streams flowing through the plurality of first flow channels are blown out, the plurality of first blowoff ports being in communication with the plurality of first flow channels and the outside of the body, the first air streams each being the first air stream; and
a plurality of second blowoff ports from which the active ingredient flowing through the plurality of second flow channels is blown out, the plurality of second blowoff ports being in communication with the plurality of second flow channels and the outside of the body, the plurality of second blowoff ports each being the second blowoff port, and
the plurality of second blowoff ports are arranged at locations where the active ingredient flowing through the plurality of second flow channels that are in communication with the plurality of second blowoff ports is blown out from the plurality of second blowoff ports by being drawn by the first air streams blown out from the plurality of first blowoff ports.

8. The active ingredient generation device according to claim 1, wherein the first flow channel narrows toward the first blowoff port in an up-down direction of the vehicle in a state where the active ingredient generation device is attached to the ceiling.

9. The active ingredient generation device according to claim 1, wherein the active ingredient generator includes a second air blower that generates a second air stream that flows through the second flow channel toward the second blowoff port, and
a power of the second air blower is lower than a power of the first air blower.

10. The active ingredient generation device according to claim 1, further comprising:
a light source that emits light for illuminating the interior of the vehicle.

11. An active ingredient generation device that is attached to a ceiling of an interior of a vehicle, the active ingredient generation device comprising:
a body;
a first air blower that is disposed inside the body and generates a first air stream;
an active ingredient generator that is disposed inside the body and generates an active ingredient;
a first flow channel through which the first air stream generated by the first air blower flows, the first flow channel being disposed inside the body; and
a second flow channel through which the active ingredient generated by the active ingredient generator flows, the second flow channel being disposed inside the body,
wherein the body includes:
a first blowoff port from which the first air stream flowing through the first flow channel is blown out, the first blowoff port being in communication with the first flow channel and an outside of the body; and
a second blowoff port from which the active ingredient flowing through the second flow channel is blown out, the second blowoff port being in communication with the second flow channel and the outside of the body,
the second blowoff port is disposed at a location where the active ingredient blown out from the second blowoff port is drawn by the first air stream blown out from the first blowoff port,
a width of the first blowoff port is smaller than a width of the first flow channel,
the body includes a plurality of first blowoff ports arranged in one direction, the plurality of first blowoff ports each being the first blowoff port, and
the plurality of first blowoff ports have different widths and are arranged in descending order of width from one side to an other side.
